(19) United States
(12) Patent Application Publication
(10) Pub. No.: US 2016/0038096 A1
(43) Pub. Date: Feb. 11, 2016

(54) METHOD AND APPARATUS FOR PROCESSING SIGNAL

(71) Applicants: Samsung Electronics Co., Ltd., Suwon-si (KR); The Industry & Academic Cooperation in Chungnam National University (IAC), Daejeon (KR)

(72) Inventors: Jong Pal KIM, Seoul (KR); Hyoung Ho KO, Daejeon (KR)

(73) Assignees: SAMSUNG ELECTRONICS CO., LTD., Suwon-si (KR); The Industry & Academic Cooperation in Chungnam National University (IAC), Daejeon (KR)

(21) Appl. No.: 14/643,172

(22) Filed: Mar. 10, 2015

(30) Foreign Application Priority Data
Aug. 11, 2014 (KR) 10-2014-0103885

(51) Int. Cl. A61B 5/00; A61B 5/024

(52) U.S. Cl. CPC A61B 5/7278; A61B 5/02427; A61B 5/725; A61B 2560/0475

(57) ABSTRACT

A signal processing method and apparatus for measuring heartbeat and oxygen saturation. The signal processing apparatus may acquire a first sampled signal by sampling a detection signal output from an optical detector in an interval in which a light source is activated, may acquire a second sampled signal by sampling a detection signal output from the optical detector in an interval in which the light source is inactivated, and may output a combined signal in which the first sampled signal and the second sampled signal are combined. An amplitude of the first sampled signal may decrease by combining the first sampled signal with the second sampled signal.

### CROSS-REFERENCE TO RELATED APPLICATION(S)

This application claims the benefit under 35 USC 119(a) of Korean Patent Application No. 10-2014-0103885, filed on Aug. 11, 2014, in the Korean Intellectual Property Office, the entire disclosure of which is incorporated herein by reference for all purposes.

### BACKGROUND

1. Field

The following description relates to signal processing technology for processing an electrical signal.

2. Description of Related Art

In general, a method of measuring a heartbeat and oxygen saturation (SpO₂) in blood vessels consists of irradiating an optical signal output from a light emitting diode (LED) toward the blood vessels and analyzing the optical signal detected at a photo diode of an optical light receiver. Here, the light receiver measures a signal in which a target signal to be measured and a background signal generated by ambient light are combined. The background signal is not measured and thus, the effect of the background signal on the measured signal is reduced enhancing the measurement accuracy.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, there is provided a signal processing apparatus including a first sampler & holder coupled to an optical detector, the first sampler & holder configured to acquire a first sampled signal by sampling a detection signal acquired in an interval in which a light source is activated, a second sampler & holder coupled to the optical detector, the second sampler & holder configured to acquire a second sampled signal by sampling a detection signal acquired in an interval in which the light source is inactivated, and a signal combiner configured to output a combined signal acquired by combining the first sampled signal and the second sampled signal.

An amplitude of the first sampled signal may decrease by combining the first sampled signal with the second sampled signal.

The signal combiner may include a node that connects a first signal line to which the first sampled signal is transferred and a second signal line to which the second sampled signal is transferred. Output terminals of the first sampler & holder and the second sampler & holder may be connected to opposite polarities.

The signal combiner may be further configured to combine the first sampled signal and the second sampled signal after sampling the second sampled signal in the interval in which the light source is inactivated.

The first sampler & holder may include a first switch configured to control sampling of the detection signal, and at least one first capacitor configured to store the first sampled signal.

The second sampler & holder may include a second switch configured to control sampling of the detection signal, and at least one second capacitor configured to store the second sampled signal.

The signal combiner may include a third switch configured to control a combination between the first sampled signal and the second sampled signal, and at least one third capacitor configured to store the combined signal.

The first sampler & holder may be further configured to acquire the first sampled signal by low pass filtering the detection signal, and by sampling the filtered detection signal.

The second sampler & holder may be further configured to acquire the second sampled signal by low pass filtering the detection signal, and by sampling the filtered detection signal.

The signal processing apparatus may further include a controller configured to control operations of the first sampler & holder and the second sampler & holder. The controller may be further configured to output a control signal for activating the light source, control the first sampler & holder to sample the first sampled signal in the interval in which the light source is activated, and control the second sampler & holder to sample the second sampled signal in the interval in which the light source is inactivated.

In another general aspect, there is provided a signal processing apparatus including a first switch coupled to an optical detector, the first switch configured to control sampling of a detection signal in an interval in which a light source is activated, a first capacitor configured to store a first sampled signal sampled by the first switch, a second switch coupled to the optical detector, the second switch configured to control sampling of the detection signal acquired in an interval in which the light source is inactivated, a second capacitor configured to store a second sampled signal sampled by the second switch, and a third switch configured to control a connection between the first capacitor and the second capacitor in the interval in which the light source is inactivated.

The first switch may be shorted in the interval in which the light source is activated, the second switch may be shorted in the interval in which the light source is inactivated, and the third switch may be shorted after the first switch and the second switch are opened in the interval in which the light source is inactivated.

The signal processing apparatus may further include a third capacitor configured to store a combined signal acquired by combining the first sampled signal and the second sampled signal.

In still another general aspect, there is provided a signal processing method including acquiring a first sampled signal by sampling a detection signal acquired in an interval in which a light source is activated, acquiring a second sampled signal by sampling a detection signal acquired in an interval in which the light source is inactivated, and outputting a combined signal acquired by combining the first sampled signal and the second sampled signal.

Other features and aspects will be apparent from the following detailed description, the drawings, and the claims.

### BRIEF DESCRIPTION OF THE DRAWINGS

FIG. 1 is a diagram illustrating an example of a signal processing apparatus.

FIG. 2 is a diagram illustrating an example of a circuit of a signal processing apparatus.

FIGS. 3 and 4 are diagrams illustrating examples in which a signal processing apparatus is utilized.

FIGS. 5 and 6 are diagrams illustrating other examples in which a signal processing apparatus is utilized.

FIG. 7 is a flowchart of a method for processing a signal according to various examples.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

Example embodiments will be described with reference to the accompanying drawings. The predetermined structural and functional descriptions in the example embodiments are provided to help the understanding of the example embodiments and thus, it is intended that the example embodiments obscure the modifications and variations of this invention provided they come within the scope of the appended claims and their equivalents. Like numbers refer to like elements throughout and known functions and structures are omitted.

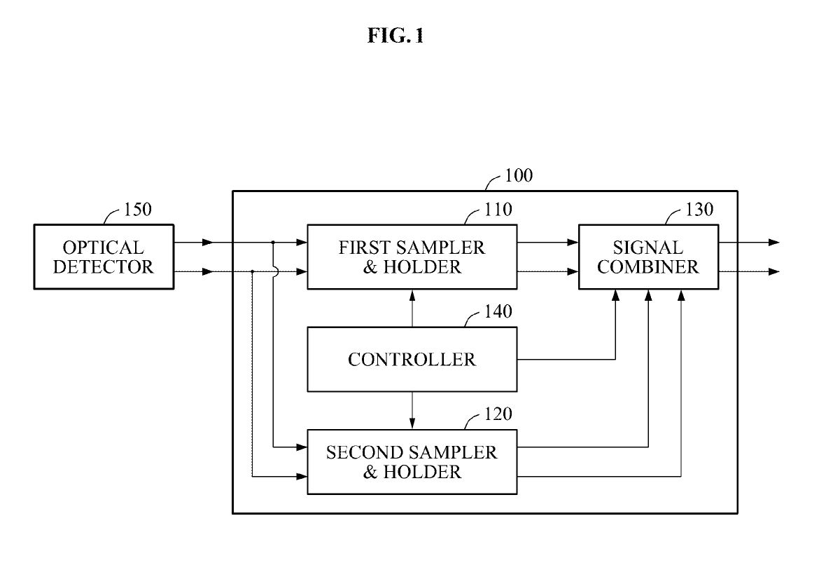

FIG. 1 is a diagram illustrating an example of a signal processing apparatus.

Referring to FIG. 1, the signal processing apparatus 100 receives a detection signal output from an optical detector 150 as an input signal. The optical detector 150 converts an incident optical signal into an electrical signal, and outputs the converted electrical signal as the detection signal. In an example, the optical detector 150 may be incorporated to operate in a measurement device configured to measure photo plethysmograph (PPG) or oxygen saturation (SpO₂) of a user based on an optical signal.

In general, to measure PPG, a light is irradiated toward a predetermined portion of a human body and blood flow is measured using an optical absorption rate absorbed in or reflected from the tissue. The PPG may be measured by measuring a state of arterial blood volume increasing and decreasing in the blood vessels at an end of a finger. The arterial blood volume increases and decreases in response to a heartbeat. When a light is irradiated from a light source toward a finger, light absorption occurs in the blood, bones, and the tissue, and a light having passed through the finger may reach the optical detector 150. An optical absorption rate is proportional to an amount of skin, tissue, and blood present in a path through which the light passes, and a change in blood flow is proportional to a change in an amount of light absorbed.

The amount of light detected at the optical detector 150 corresponds to the amount of light transmitted from the finger. That is, the amount of light received at the optical detector 150 is the amount of light transmitted to the finger minus an amount of light absorbed at the finger. Thus, a change in an amount of the transmitted light detected at the optical detector 150 represents a change in blood flow. A change in a blood amount synchronized with a heartbeat may be estimated based on the change in the amount of light detected at the optical detector 150. A time between peaks may be calculated based on the estimated change in the blood amount and the heartbeat may be estimated based on the calculated time.

The light irradiated from the light source is incident to the optical detector 150. Also, a background signal generated by an ambient light is incident to the optical detector 150. When a target signal to be measured and the background signal are incident to the optical detector 150, the background signal acts as noise. Therefore, a signal-to-noise ratio (SNR) decreases according to an increase of the background signal, which makes an accurate measurement difficult. In addition, when the background signal is not removed from the detection signal, an output signal is saturated due to the background signal and the target signal to be measured may not be properly amplified.

The signal processing apparatus 100 removes the background signal from the detection signal received by the optical detector 150. The signal processing apparatus 100 may remove the background signal from a pulse-type input signal that operates as an optical signal. For example, the signal processing apparatus 100 may enhance an SNR by removing a background signal from a detection signal when amplifying a pulse-type input that operates as an optical signal such as pulse oximeter or oxygen saturation.

The signal processing apparatus 100 may be used as part of a variety of portable medical/health devices, a smartphone, and a tablet personal computer (PC). However, the use of the signal processing apparatus 100 is not limited thereto and thus may be used with any other electronic device. For example, the signal processing apparatus 100 may be used in a medical device and a bio-health monitoring device capable of measuring a heartbeat or oxygen saturation in the blood vessel using an optical signal.

Referring to FIG. 1, the signal processing apparatus 100 includes a first sampler & holder 110, a second sampler & holder 120, a signal combiner 130, and a controller 140.

The signal processing apparatus 100 samples a detection signal output from the optical detector 150 in an interval in which a light source (not shown) irradiates light and is thus activated or ON and in an interval in which the light source does not irradiate light and is thus inactivated or OFF. The signal processing apparatus 100 acquires a first sampled signal by sampling and holding the detection signal output from the optical detector 150 in a state in which the light source is activated, and acquires a second sampled signal by sampling and holding the detection signal output from the optical detector 150 in a state in which the light source is inactivated.

The first sampler & holder 110 acquires the first sampled signal by sampling the detection signal coupled to the optical detector 150 in the interval in which the light source is activated. The first sampler & holder 110 includes a first switch configured to control whether to sample the detection signal output from the optical detector 150 and at least one first capacitor configured to store the first sampled signal. The shorting and opening of the first switch is controlled by the controller 140, and the detection signal is sampled when the first switch is shorted. The first sampled signal may include a target signal to be measured and a background signal generated by an ambient light. In the interval in which the light source is activated, the second sampler & holder 120 is disconnected from the optical detector 150 and does not sample the detection signal.

In an example, the first sampler & holder 110 may acquire the first sampled signal by low pass filtering the detection signal and sampling the filtered detection signal. For example, the first sampler & holder 110 may include a circuit configuration in which a first resistor is connected between the first switch and the first capacitor and one end of the first capacitor is connected to a ground (GND). Here, the first capacitor may operate as a sampling capacitor and may be connected to the first resistor and operate as an RC low pass filter.

The second sampler & holder 120 acquires the second sampled signal by sampling the detection signal coupled to the optical detector 150 in the interval in which the light source is OFF or inactivated. The second sampler & holder 120 is connected to the first sampler & holder 110 in parallel. The second sampler & holder 120 includes a second switch configured to control whether to sample the detection signal output from the optical detector 150 and at least one second capacitor configured to store the second sampled signal. The short and open of the second switch is controlled by the controller 140, and the detection signal is sampled when the second switch is shorted. In a state in which the light source is OFF or inactivated, an external light is incident to the optical detector 150 and the optical detector 150 outputs the detection signal with respect to the external light. Accordingly, the second sampled signal generated by sampling the detection signal may include the background generated by the external light. In the interval in which the light source is OFF or inactivated, the first sampler & holder 110 is disconnected from the optical detector 150 and does not sample the detection signal.

In an example, the second sampler & holder 120 may acquire the second sampled signal by low pass filtering the detection signal and sampling the filtered detection signal. For example, the second sampler & holder 120 may include a circuit configuration in which a second resistor is connected between the second switch and the second capacitor and one end of the second capacitor is connected to the GND. Here, the second capacitor may operate as a sampling capacitor and may be connected to the second resistance and operate as an RC band pass filter.

The signal combiner 130 combines the first sampled signal and the second sampled signal that are analog signals, and outputs a combined signal in which the first sampled signal and the second sampled signal are combined. An amplitude of the first sampled signal decreases by being combined with the second sampled signal. The background signal included in the first sampled signal is removed by combining the background signal included in the first sampled signal with the background signal included in the second sampled signal. The target signal in which the background signal is removed is output as an output signal of the signal processing apparatus 100.

When the second sampled signal is sampled in the interval in which the light source is OFF or inactivated, the signal combiner 130 combines the first sampled signal and the second sampled signal. The signal combiner 130 combines the first sampled signal and the second sampled signal in an interval in which the first sampler & holder 110 and the second sampler & holder 120 are disconnected from the optical detector 150.

The signal combiner 130 includes a node that connects a first signal line to which the first sampled signal is transferred and a second signal line to which the second sampled signal is transferred. Output terminals of the first sampler & holder 110 and the second sampler & holder 120 may be connected to opposite polarities. For example, in the node, + polarity of the first signal line may be connected to − polarity of the second signal line and − polarity of the first signal line may be connected to + polarity of the second signal line. Accordingly, an amplitude of the first sampled signal may decrease by combining the first sampled signal with the second sampled signal.

The signal combiner 130 includes a plurality of third switches configured to control a combination between the first sampled signal and the second sampled signal and at least one third capacitor configured to store a combined signal between the first sampled signal and the second sampled signal. In an example, each of the third switches may be connected between the first capacitor and the node and between the second capacitor and the node. The third capacitor may be connected between the third switches and an output end of the signal processing apparatus 100.

The controller 140 controls operations of the light source, the first sampler & holder 110, and the second sampler & holder 120. The controller 140 controls whether to activate the light source using a control signal. The controller 140 controls the first sampler & holder 110 to sample the first sampled signal in the interval in which the light source is ON or activated, and controls the second sampler & holder 120 to sample the second sampled signal in the interval in which the light source is OFF or inactivated. The controller 140 controls whether to short and open the first switch, the second switch, and the third switch using a switching control signal. Switching control signals for controlling the respective switches may non-overlap on a temporal axis.

The signal processing apparatus 100 outputs a combined signal in which the first sampled signal and the second sampled signal are combined. The combined signal includes a target signal in which the background signal is removed from the first sampled signal. The signal processing apparatus 100 may have a reduced design area since it does not use a differential amplifier to remove the background signal from the detection signal, and is free from a constraint in that only a background signal less than the input range of the differential amplifier is removable. Also, the signal processing apparatus 100 may remove the background signal using an analog method and may normally remove the background signal from the detection signal regardless of the intensity of the ambient light incident to the optical detector 150.

The combined signal output from the signal processing apparatus 100 is amplified by an amplifier (not shown), and is converted to a digital signal by an analog-to-digital (A/D) converter (not shown).

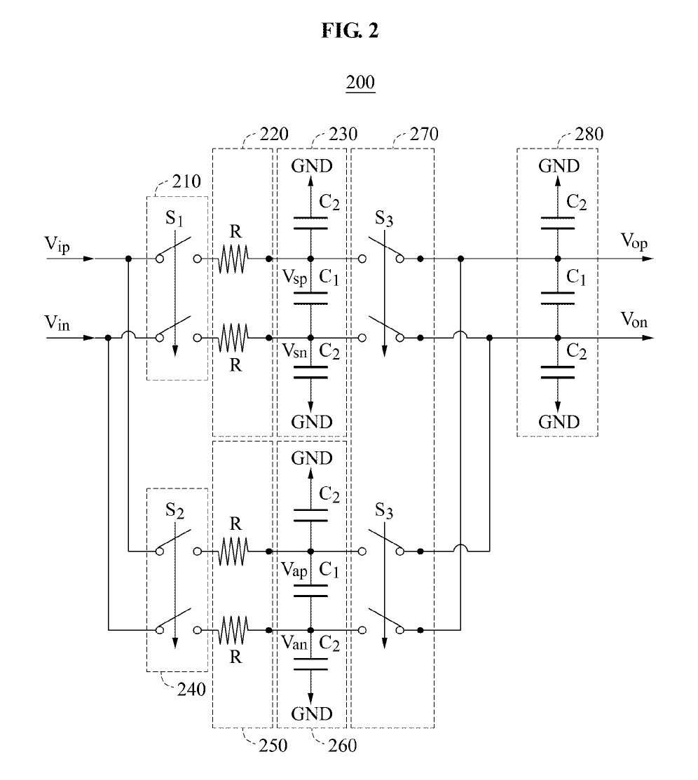

FIG. 2 illustrates an example of a circuit of a signal processing apparatus.

Referring to FIG. 2, the signal processing apparatus 200 removes a background signal from an input signal using a sample & hold circuit. For example, the signal processing apparatus 200 is connected to an optical detector (not shown) and configured to receive detection signals Vᵢₚ and Vᵢₙ output from the optical detector through an input terminal. The signal processing apparatus 200 includes a first sampler & holder, a second sampler & holder, and a signal combiner. The first sampler & holder and the second sampler & holder are connected in parallel.

The first sampler & holder includes first switches 210, first resistances 220, and first capacitors 230. The first switches 210 control whether to sample a detection signal output from the optical detector in an interval in which a light source is ON or activated. The first switches 210 are connected between the input terminal of the signal processing apparatus 200 and the first resistances 220. The first capacitors 230 store or hold a first sampled signal sampled by the first switches 210. The first capacitors 230 include a capacitor configured to maintain a common mode signal when sampling a detection signal. The first capacitors 230 may constitute an RC low pass filter together with the first resistances 220. The filtered first sampled signal may be stored in the first capacitors 230.

The second sampler & holder includes second switches 240, second resistances 250, and second capacitors 260. The second switches 240 control whether to sample a detection signal output from the optical detector in an interval in which the light source is OFF or inactivated. The second switches 240 are connected between the input terminal of the signal processing apparatus 200 and the second resistances 250. The second capacitors 260 store or hold a second sampled signal sampled by the second switches 240. The second capacitors 260 include a capacitor configured to maintain a common mode signal when sampling a detection signal. The second capacitors 260 may constitute an RC low pass filter together with the second resistances 250. The filtered second sampled signal may be stored in the second capacitors 260.

The signal combiner includes third switches 270 and third capacitors 280. The third switches 270 control connections between the first capacitors 230 and the second capacitors 260. When the third switches 270 are shorted, the first capacitors 230 are connected to the second capacitors 260, and the first sampled signal stored in the first capacitors 230 and the second sampled signal stored in the second capacitors 260 may be combined. The first capacitors 230 and the second capacitors 260 may be cross-connected. Through redistribution of charges stored in the first capacitors 230 and the second capacitors 260, a target signal in which a background signal is removed may be extracted. The third capacitors 280 may store a combined signal in which the first sampled signal and the second sampled signal are combined. The third capacitors 280 maintain a common mode signal and a differential mode signal of a combined signal. The combined signal stored in the third capacitors 280 may be output through an output terminal of the signal processing apparatus 200 as output signals Vₒₚ and Vₒₙ.

In a first time interval in which the light source is ON or activated, the first switches 210 may be shorted, and the second switches 240 and the third switches 270 may be opened. In this example, the first resistances 220 and the first capacitors 230 may constitute an RC low pass filter. In input signals Vᵢₚ and Vᵢₙ, frequency components less than a cutoff frequency of the RC low pass filter including the first resistances 220 and the first capacitors 230 may be stored in the first capacitors 230. Here, the first sampled signal stored in the first capacitors 230 after the input signals Vᵢₚ and Vᵢₙ are sampled and defined as Vₛₚ and Vₛₙ, respectively, and V_{signal}=Vₛₚ−Vₛₙ is defined.

In a second time interval that is a subsequent time interval to the first time interval, the first switches 210 may be opened and the second switches 240 may be shorted. When the first switches 210 are opened, the second switches 240 may be shorted. That is, the first switches 210 and the second switches 240 are not in a short state concurrently. The third switches 270 may maintain an open state, which is the same as in the first time interval. In the second time interval, the light source is OFF or inactivated and an ambient light may be incident to the optical detector.

The input signals Vᵢₚ and Vᵢₙ are sampled through the RC low pass filter including the second resistances 250 and the second capacitors 260, and a sampled signal may be stored in the second capacitors 260. Here, the second sampled signal stored in the second capacitors 260 after the input signals Vᵢₚ and Vᵢₙ are sampled are defined as Vₐₚ and Vₐₙ, respectively, and V_{ambient}=Vₐₚ−Vₐₙ is defined.

In a third time interval that is a subsequent time interval to the second time interval, the second switches 240 may be opened and the third switches 270 may be shorted. When the second switches 240 are opened, the third switches 270 may be shorted. That is, the second switches 240 and the third switches 270 are not in a short state concurrently. The first switches 210 may maintain an open state, which is the same as in the second time interval. In the third time interval, the light source may be OFF or in an inactive state, which is the same as in the second time interval.

When the third switches 270 are shorted, the first capacitors 230 and the second capacitors 260 may be connected to each other, and charges stored in the respective capacitors 230 and 260 may be redistributed to the third capacitors 280 based on a charge conservation rule. Through charge redistribution, the first sampled signal sampled to the first capacitors 230 by a switching operation of the first switches 210 and the second sampled signal sampled to the second capacitors 260 by a switching operation of the second switches 240 may be combined with each other. The first switches 210, the second switches 240, and the third switches 270 may be sequentially connected, and may be driven by a non-overlapping clock.

The combined signal between the first sampled signal and the second sampled signal that are signals redistributed by the third capacitors 280 are defined as Vₒₚ[n] and Vₒₙ[n]. Signals stored in the third capacitors 280 before the third switches 270 are shorted are defined as Vₒₚ[n−1] and Vₒₙ[n−1]. Additionally, charges stored in the third capacitors 280 may be conserved in each of a case in which the third switches 270 are opened and in a case in which the third switches 270 are shorted. Accordingly, the following relationships may be achieved as expressed by Equation 1 and Equation 2.

C₂Vₛₚ+C₁(Vₛₚ−Vₛₙ)+C₁(Vₐₙ−Vₐₚ)+C₂Vₐₙ+C₂Vₒₚ[n−1]+C₁(Vₒₚ[n−1]−Vₒₙ[n−1))=3C₂Vₒₚ+3C₁(Vₒₚ−Vₒₙ) [Equation 1]

C₂Vₛₙ+C₁(Vₛₙ−Vₛₚ)+C₁(Vₐₚ−Vₐₙ)+C₂Vₐₚ+C₂Vₒₙ[n−1]+C₁(Vₒₙ[n−1]−Vₒₚ[n−1))=3C₂Vₒₙ+3C₁(Vₒₙ−Vₒₚ) [Equation 2]

In Equation 1 and 2, Vₛₚ and Vₛₙ denote the first sampled signals stored in the first capacitors 230, Vₐₚ and Vₐₙ denote the second sampled signals stored in the second capacitors 260, C₁ denotes a capacitance of a capacitor connected between differential signal lines among the third capacitors 280, and C₂ denotes a capacitance of a capacitor between any one differential signal line among the third capacitors 280 and a ground.

When an input signal is assumed to vary slowly, relationships of Equation 3 and Equation 4 may be assumed.

Vₒₚ≈Vₒₚ[n]≦Vₒₚ[n−1] [Equation 3]

Vₒₙ≈Vₒₙ[n]≦Vₒₙ[n−1] [Equation 4]

Based on the relationships of Equation 3 and Equation 4 by subtracting a result of Equation 2 from a result of Equation 1, a differential signal Vₒᵤₜ output from the signal processing apparatus 200 may be expressed by Equation 5.

$${V}_{out} = {V}_{op} - {V}_{on} = \frac{1}{2} ( ( {V}_{sp} - {V}_{sn} ) - ( {V}_{ap} - {V}_{an} ) ) = \frac{1}{2} ( {V}_{signal} - {V}_{ambient} ) \qquad [ Equation 5 ]$$

From a result of Equation 5, it can be known that the differential signal Vₒᵤₜ=(Vₒₚ−Vₒₙ) output from the output terminal of the signal processing apparatus 200 indicates a signal in which a background signal is removed and only a target signal to be substantially measured is included.

In an example, while the third switches 270 are being opened, the signal processing apparatus 200 may perform one of the following two methods to set charges stored in the third capacitors 280 to be in a constant state. As a first method, a fourth switch (not shown) is present between two nodes of the third capacitors 280, for examples, nodes to which the third switches 270 and the third capacitors 280 are connected, and the signal processing apparatus 200 may control a differential potential difference to be absent between the two nodes of the third capacitors 280 by shorting the fourth switch during a predetermined period of time in an interval in which the third switches 270 are opened. As a second method, a fifth switch (not shown) and a sixth switch (not shown) are present between two nodes of the third capacitors, that is, nodes to which the third switches 270 and the third capacitors 280 are connected, and the ground, respectively, and the signal processing apparatus 200 may set the two nodes of the third capacitors 280 to a ground voltage by shorting the fifth switch and the sixth switch during a predetermined period of time in the interval in which the third switches 270 are opened.

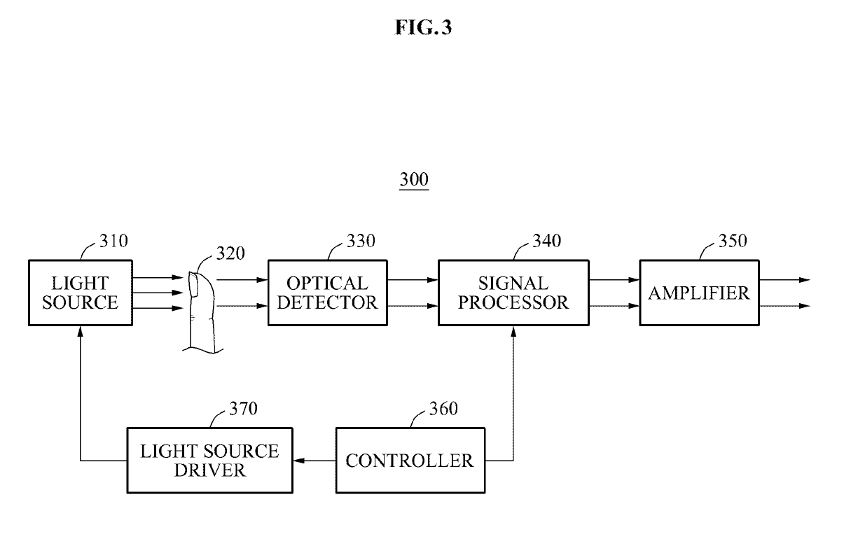

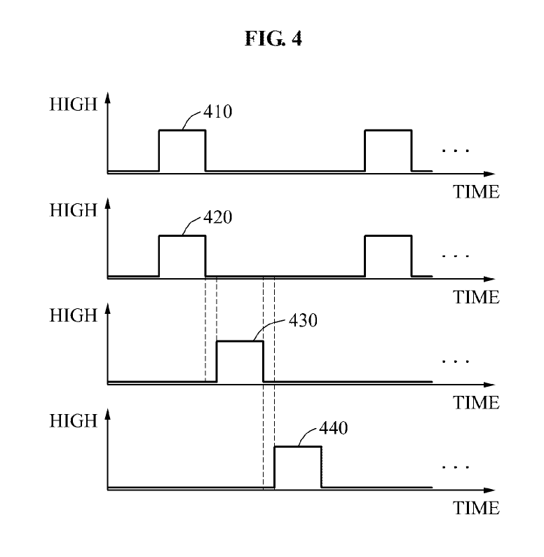

FIGS. 3 and 4 are diagrams illustrating examples in which a signal processing apparatus is utilized.

FIG. 3 illustrates an example of a pulse wave measurement system 300 using PPG. Referring to FIG. 3, the pulse wave measurement system 300 includes a light source 310, an optical detector 330, a signal processor 340, an amplifier 350, a controller 360, and a light source driver 370. In an embodiment, the pulse wave measurement system 300 may irradiate light toward a measurement target 320 such as a finger of a user, for example, and may measure light reflected from the measurement target 320 or light having passed through the measurement target 320, using the optical detector 330.

The signal processor 340 removes a background signal from a detection signal output from the optical detector 330. The signal processor 340 may sample the detection signal independently in an interval in which the light source 310 is ON or activated and irradiates the light and the signal processor 340 may sample the detection signal in an interval in which the light source 310 is OFF or inactivated and does not irradiate the light.

Hereinafter, a process in which the signal processor 340 removes the background signal from the detection signal transferred from the optical detector 330 will be described with reference to the circuit of FIG. 2 and a timing diagram of FIG. 4. The signal processor 340 may correspond to the signal processing apparatus 200 of FIG. 2.

Referring to FIG. 4, the controller 360 generates control signals 410, 420, 430, and 440. The controller 360 applies the control signal 410 for controlling the light source 310 to the light source driver 370. When the control signal 410 is a high signal, the light source driver 370 may activate the light source 310 by applying a current to the light source 310. The light source 310 may irradiate a light toward the measurement target 320 in an active state.

The optical detector 330 detects transmitted light having passed through the measurement target 320 or light reflected from the measurement target 320. The optical detector 330 converts the detected transmitted light or reflected light to an electrical signal using a photodiode (not shown) and a trans-impedance amplifier (not shown).

In an interval in which the light source 310 is ON or activated, that is, in an interval in which the control signal 410 is a high signal, the high signal is applied to the control signal 420 for controlling first switches and the first switches are shorted. In an interval in which the light source 310 is ON or activated, a target signal to be measured and a background signal by an ambient light may be included in a detection signal transferred from the optical detector 330. When the first switches are shorted, a first sampled signal including the target signal and the background signal may be sampled to first capacitors.

When a low signal is applied to the control signal 410, the light source driver 370 inactivates the light source 310. When the low signal is applied to the control signal 410, the low signal may also be applied to the control signal 420 for controlling the first switches. When the light source 310 is OFF or inactivated, a high signal is applied to the control signal 430 for controlling second switches and the second switches are shorted. In an interval in which the light source 310 is OFF or inactivated, only a background signal by an ambient light may be included in a detection signal transferred from the optical detector 330. When the second switches are shorted, a second sampled signal including the background signal may be sampled to second capacitors.

When a low signal is applied to the control signal 430 and the second switches are opened, a high signal may be applied to the control signal 440 for controlling third switches. The control signals 420, 430, and 440 for controlling the respective switches do not overlap on a temporal axis and do not overlap each other in a high state.

When the high signal is applied to the control signal 440, the third switches are shorted and the first sampled signal stored in the first capacitors and the second sampled signal stored in the second capacitors may be combined. Polarities of the first capacitors and the second capacitors are cross-connected to each other. An amplitude of the first sampled signal may decrease by being combined with the second sampled signal. The background signal included in the first sampled signal is removed by the background signal included in the second sampled signal. Only the target signal to be measured may be stored in the third capacitors.

The target signal stored in the third capacitors may be output through the output terminal of the signal processor 340 and may be input to the amplifier 350. For example, the amplifier 350 may be a programmable gain amplifier. The amplifier 350 amplifies the target signal transferred from the signal processor 340. The signal processor 340 removes a background signal from a detection signal, thereby enhancing a signal-to-noise ratio (SNR) and preventing an input signal having a level beyond the allowable range from being input to the amplifier 350.

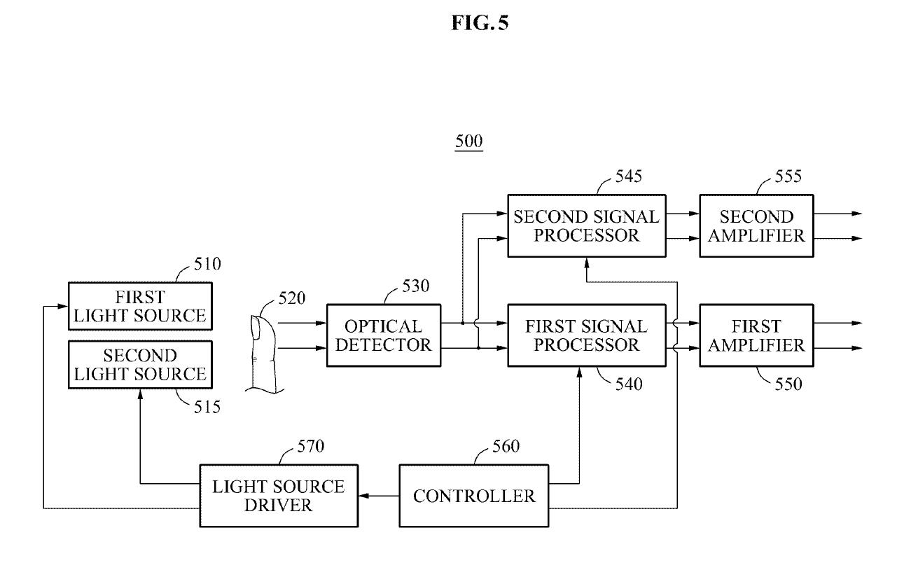

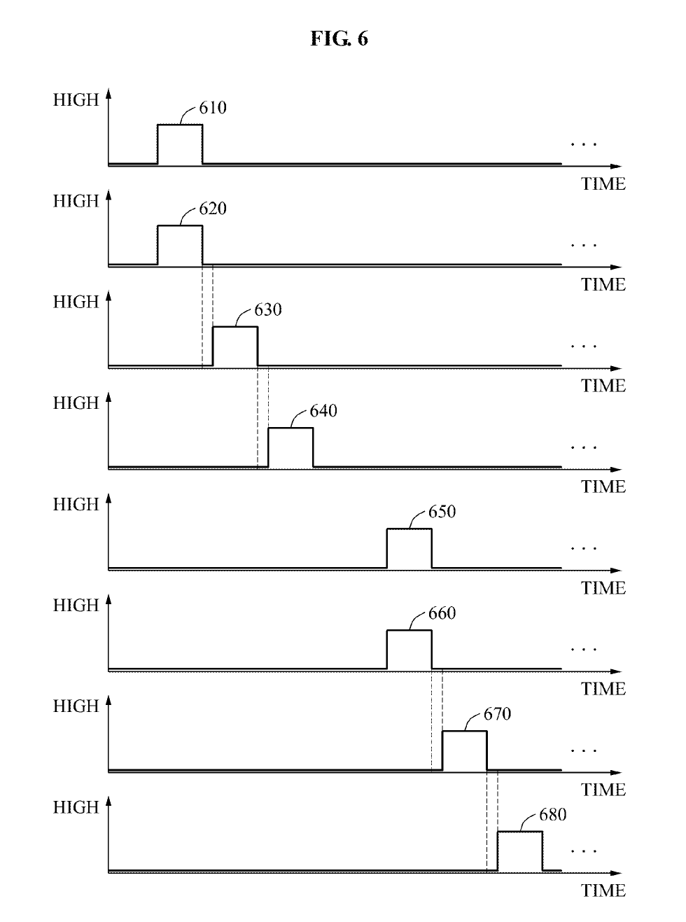

FIGS. 5 and 6 are diagrams illustrating other examples in which a signal processing apparatus is utilized.

FIG. 5 illustrates an example of an oxygen saturation measurement system 500. Referring to FIG. 5, the oxygen saturation measurement system 500 includes a first light source 510, a second light source 515, an optical detector 530, a first signal processor 540, a second signal processor 545, a first amplifier 550, a second amplifier 555, a controller 560, and a light source driver 570. In an example, the oxygen saturation measurement system 500 may measure an oxygen saturation of a measurement target 520 using the first light source 510 configured to irradiate a red light and the second light source 515 configured to irradiate an infrared (IR) light.

The oxygen saturation measurement system 500 acquires a first detection signal by irradiating the red light toward the measurement target 520 such as a finger of a user, for example, and by measuring the light reflected from the measurement target 520 or having passed through the measurement target 520, using the optical detector 530. The oxygen saturation measurement system 500 acquires a second detection signal by irradiating the IR light toward the measurement target 520 and by measuring the light reflected from the measurement target 520 or having passed through the measurement target 520, using the optical detector 530. The oxygen saturation measurement system 500 may measure the oxygen saturation based on a difference between an optical absorption rate by a wavelength of the red light and an optical absorption rate by a wavelength by the IR light.

Each of the first signal processor 540 and the second signal processor 545 removes a background signal from the detection signal coupled to the optical detector 530. The first signal processor 540 may sample the detection signal independently in an interval in which the first light source 510 is ON or activated and irradiates the red light and in an interval in which the first light source 510 is OFF or inactivated and does not irradiate the red light. The second signal processor 545 may sample the detection signal independently in an interval in which the second light source 515 is ON or activated and irradiates the IR light and in an interval in which the second light source 515 is OFF or inactivated and does not irradiate the IR light.

Hereinafter, a process in which each of the first signal processor 540 and the second signal processor 545 removes the background signal from the detection signal transferred from the optical detector 530 will be described with reference to the circuit of FIG. 2 and a timing diagram of FIG. 6. Each of the first signal processor 540 and the second signal processor 545 may correspond to the signal processing apparatus 200 of FIG. 2.

Referring to FIG. 6, the controller 560 generates control signals 610, 620, 630, 640, 650, 660, 670, and 680. The controller 560 applies the control signal 610 to the light source driver 570 for controlling the first light source 510 or applies the control signal 650 to the light source driver 570 for controlling the second light source 515. When the control signal 610 is a high signal, the light source driver 570 may activate the first light source 510 by applying a current to the first light source 510. The first light source 510 may irradiate a red light toward the measurement target 520 in an active state. When the control signal 650 is a high signal, the light source driver 570 may activate the second light source 515 by applying a current to the second light source 515. The second light source 515 may irradiate an IR light toward the measurement target 520 in an active state.

In an interval in which the first light source 510 is ON or activated and the second light source 515 is OFF or inactivated, the optical detector 530 may convert, to an electrical signal, the optical current generated based on the red light, using a photodiode (not shown) and a trans-impedance amplifier (not shown).

In the interval in which the first light source 510 is ON or activated and the second light source 515 is OFF or inactivated, that is, in an interval in which the control signal 610 is a high signal and the control signal 650 is a low signal, the high signal is applied to the control signal 620 for controlling the first switches included in the first signal processor 540 and the first switches are shorted. A target signal that is an optical signal with respect to the red light and a background signal by an ambient light may be included in a detection signal output from the optical detector 530. When the first switches are shorted, a first sampled signal each including the target signal and the background signal may be sampled to first capacitors.

When a low signal is applied to the control signal 610, the light source driver 570 inactivates the first light source 510. When the low signal is applied to the control signal 610, the low signal may be applied to the control signal 620 for controlling the first switches of the first signal processor 540. When the first light source 510 is OFF or inactivated, the high signal may be applied to the control signal 630 for controlling second switches of the first signal processor 540, and the second switches may be shorted. In an interval in which the first light source 510 is OFF or inactivated, only the background signal generated by the ambient light may be detected by the optical detector 530. When the second switches are shorted, a second sampled signal including the background signal may be sampled to the second capacitors.

When a low signal is applied to the control signal 630 and the second switches are opened, a high signal may be applied to the control signal 640 for controlling third switches of the first signal processor 540. When the high signal is applied to the control signal 640, the third switches are shorted and the first sampled signal stored in the first capacitors and the second sampled signal stored in the second capacitors may be combined. A combined signal in which the first sampled signal and the second sampled signal are combined may be amplified by the first amplifier 550.

When the first light source 510 is OFF or inactivated and when a measurement based on the IR light of the second light source 515 starts based on the control signal 650, the same process as the process performed by the first signal processor 540 may be performed by the second signal processor 545. The second signal processor 545 may remove a background signal from a signal detected based on the IR light, based on the control signal 660 for controlling the first switch, the control signal 670 for controlling the second switch, and the control signal 680 for controlling the third switch.

In an interval in which the second light source 515 is activated, a high signal may be applied to the control signal 660 and an optical signal by the IR light and the background signal may be sampled. When the high signal is applied to the control signal 670 and the background signal is sampled, a combined signal in which the background signal is removed may be generated through charge redistribution in an interval in which the control signal 680 is a high signal. The combined signal may be amplified by the second amplifier 555. The oxygen saturation may be calculated based on a rate between an output signal of the first amplifier 550 and an output signal of the second amplifier 555.

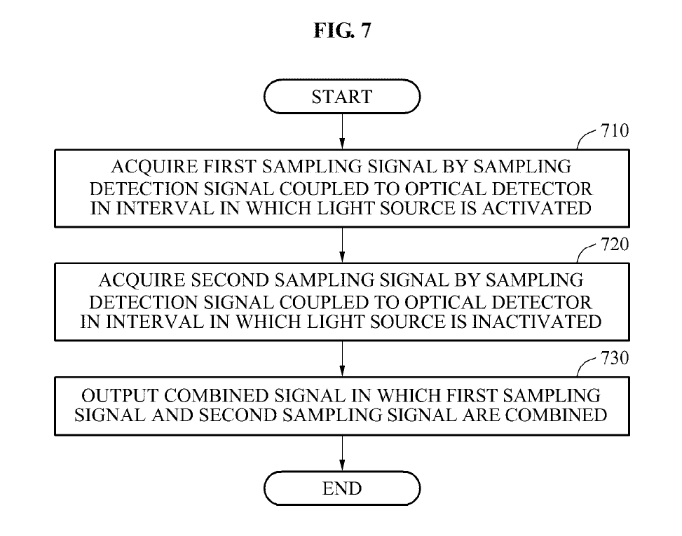

FIG. 7 is a flowchart of a method for processing a signal according to various examples.

In operation 710, in an interval in which a light source is ON or activated, a signal processing apparatus acquires a first sampled signal by sampling a detection signal coupled to an optical detector. The first sampled signal may include a target signal and a background signal generated by ambient light. In an example, the signal processing apparatus may acquire the first sampled signal by low pass filtering the detection signal output from the optical detector and sampling the filtered detection signal.

In operation 720, in an interval in which the light source is OFF or inactivated, the signal processing apparatus acquires a second sampled signal by sampling the detection signal coupled to the optical detector. In a state in which the light source is inactivated, an external light is incident to the optical detector and the optical detector outputs the detection signal with respect to the external light. The second sampled signal may include a background signal generated by the external light. In an example, the signal processing apparatus may acquire the second sampled signal by low pass filtering the detection signal output from the optical detector and sampling the filtered detection signal.

In operation 730, the signal processing apparatus outputs a combined signal in which the first sampled signal and the second sampled signal are combined. An amplitude of the first sampled signal decreases by being combined with the second sampled signal. In the interval in which the light source is OFF or inactivated, when the second sampled signal is sampled, the signal processing apparatus may combine the first sampled signal and the second sampled signal. The background signal included in the first sampled signal is removed by being combined with the background signal included in the second sampled signal and the combined signal may include only a target signal. An output signal output from the signal processing apparatus may be amplified by an amplifier and may be converted to a digital signal by an A/D converter.

The units described herein may be implemented using hardware components and software components. For example, the hardware components may include microphones, amplifiers, band-pass filters, audio to digital converters, and processing devices. A processing device may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The processing device may nm an operating system (OS) and one or more software applications that run on the OS. The processing device also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a processing device may include multiple processors or a processor and a controller. In addition, different processing configurations are possible, such a parallel processors.

The software may include a computer program, a piece of code, an instruction, or some combination thereof, to independently or collectively instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network combined computer systems so that the software is stored and executed in a distributed fashion. The software and data may be stored by one or more non-transitory computer readable recording mediums.

The non-transitory computer readable recording medium may include any data storage device that can store data which can be thereafter read by a computer system or processing device. Examples of the non-transitory computer readable recording medium include read-only memory (ROM), random-access memory (RAM), CD-ROMs, magnetic tapes, floppy disks, and optical data storage devices. Also, functional programs, codes, and code segments that accomplish the examples disclosed herein can be easily construed by programmers skilled in the art to which the examples pertain based on and using the flow diagrams and block diagrams of the figures and their corresponding descriptions as provided herein.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A signal processing apparatus comprising:
a first sampler & holder coupled to an optical detector, the first sampler & holder configured to acquire a first sampled signal by sampling a detection signal acquired in an interval in which a light source is activated;
a second sampler & holder coupled to the optical detector, the second sampler & holder configured to acquire a second sampled signal by sampling a detection signal acquired in an interval in which the light source is inactivated; and
a signal combiner configured to output a combined signal acquired by combining the first sampled signal and the second sampled signal,
wherein an amplitude of the first sampled signal decreases by combining the first sampled signal with the second sampled signal.

2. The signal processing apparatus of claim 1, wherein the first sampler & holder and the second sampler & holder are connected in parallel, and
output terminals of the first sampler & holder and the second sampler & holder are connected to opposite polarities.

3. The signal processing apparatus of claim 1, wherein the signal combiner is further configured to combine the first sampled signal and the second sampled signal after sampling the second sampled signal in the interval in which the light source is inactivated.

4. The signal processing apparatus of claim 1, wherein the first sampler & holder comprises:
a first switch configured to control sampling of the detection signal acquired in the interval in which the light source is activated; and
at least one first capacitor configured to store the first sampled signal.

5. The signal processing apparatus of claim 1, wherein the second sampler & holder comprises:
a second switch configured to control sampling of the detection signal acquired in the interval in which the light source is inactivated; and
at least one second capacitor configured to store the second sampled signal.

6. The signal processing apparatus of claim 1, wherein the signal combiner is configured to combine the first sampled signal and the second sampled signal in an interval in which the first sampler & holder and the second sampler & holder are disconnected from the optical detector.

7. The signal processing apparatus of claim 1, wherein the signal combiner comprises:
a third switch configured to control combination between the first sampled signal and the second sampled signal; and
at least one third capacitor configured to store the combined signal acquired by combining the first sampled signal and the second sampled signal.

8. The signal processing apparatus of claim 1, wherein the first sampler & holder is disconnected from the optical detector in the interval in which the light source is inactivated, and
the second sampler & holder is disconnected from the optical detector in the interval in which the light source is activated.

9. The signal processing apparatus of claim 1, wherein the first sampler & holder is further configured to acquire the first sampled signal by low pass filtering the detection signal acquired in an interval in which the light source is activated, and by sampling the filtered detection signal.

10. The signal processing apparatus of claim 1, wherein the second sampler & holder is further configured to acquire the second sampled signal by low pass filtering the detection signal acquired in the interval in which the light source is inactivated, and by sampling the filtered detection signal.

11. The signal processing apparatus of claim 1, further comprising:
a controller configured to control operations of the first sampler & holder and the second sampler & holder.

12. The signal processing apparatus of claim 11, wherein the controller is further configured to
output a control signal for activating the light source,
control the first sampler & holder to sample the first sampled signal in the interval in which the light source is activated, and
control the second sampler & holder to sample the second sampled signal in the interval in which the light source is inactivated.

13. The signal processing apparatus of claim 1, wherein the first sampled signal comprises a target signal to be measured and a background signal generated by ambient light, and
the second sampled signal comprises the background signal.

14. The signal processing apparatus of claim 13, wherein the background signal comprised in the first sampled signal is removed by combining the background signal comprised in the first sampled signal with the background signal comprised in the second sampled signal.

15. The signal processing apparatus of claim 1, wherein the optical detector is further configured to convert an incident optical signal to an electrical signal, and to output the electrical signal as a detection signal.

16. A signal processing apparatus comprising:
a first switch coupled to an optical detector, the first switch configured to control sampling of a detection signal acquired in an interval in which a light source is activated;
a first capacitor configured to store a first sampled signal sampled by the first switch;
a second switch coupled to the optical detector, the second switch configured to control sampling of the detection signal acquired in an interval in which the light source is inactivated;
a second capacitor configured to store a second sampled signal sampled by the second switch; and
a third switch configured to control a connection between the first capacitor and the second capacitor in the interval in which the light source is inactivated.

17. The signal processing apparatus of claim 16, wherein
the first switch is shorted in the interval in which the light source is activated,
the second switch is shorted in the interval in which the light source is inactivated, and
the third switch is shorted after the first switch and the second switch are opened in the interval in which the light source is inactivated.

18. The signal processing apparatus of claim 16, further comprising:
a third capacitor configured to store a combined signal acquired by combining the first sampled signal and the second sampled signal.

19. A pulse wave measurement apparatus comprising:
a light source configured to irradiate light toward a target;
a controller configured to apply control signals to drive the light source;
an optical detector configured to measure light reflected from the target and to output a detected signal; and
a signal processor configured to remove background signal from the detected signal and configured to output a target signal,
wherein the background signal is removed from the detected signal by sampling the detected signal when the light source is activated and when the light source is not activated.
